(19) United States
(12) Patent Application Publication
(10) Pub. No.: US 2021/0163372 A1
(43) Pub. Date: Jun. 3, 2021

(54) SENSOR ELEMENT

(71) Applicant: NGK INSULATORS, LTD., Nagoya-City (JP)

(72) Inventors: Kousuke UJIHARA, Tokai-City (JP); Megumi FUJISAKI, Kuwana-City (JP); Takahiro TOMITA, Chita-City (JP)

(73) Assignee: NGK INSULATORS, LTD., Nagoya-City (JP)

(21) Appl. No.: 17/172,493

(22) Filed: Feb. 10, 2021

Related U.S. Application Data
(63) Continuation of application No. PCT/JP2019/036196, filed on Sep. 13, 2019.

(30) Foreign Application Priority Data
Sep. 28, 2018 (JP) PCT/JP2018/036239

(51) Int. Cl. C04B 41/87; C04B 41/00; C04B 41/50; G01N 27/409; G01N 27/416

(52) U.S. Cl. CPC C04B 41/87; C04B 41/009; G01N 27/416; G01N 27/409; C04B 41/5031

(57) ABSTRACT

A sensor element includes a ceramic layered body having a zirconia layer part and two alumina layer parts provided on both surfaces of the zirconia layer part, respectively, and a plurality of electrodes provided in the ceramic layered body. At least one of the two alumina layer parts contains Ti element, the zirconia layer part has a layer containing Zr element and Ti element in the vicinity of an interface with the at least one alumina layer part, and the layer contains Ti element in an amount from 0.05 to 5.0 mass %.

### CROSS-REFERENCE TO RELATED APPLICATION

The present application is a continuation application of International Application No. PCT/JP2019/036196 filed on Sep. 13, 2019, which claims priority to International Application No. PCT/JP2018/036239 filed on Sep. 28, 2018. The contents of these applications are incorporated herein by reference in their entirety.

### TECHNICAL FIELD

The present invention relates to a sensor element.

### BACKGROUND ART

Conventionally, sensors using zirconia have been used. U.S. Pat. No. 5,104,744, for example, discloses a gas sensor element in which a zirconia filling part formed of a zirconia material is provided in a filling through hole provided in an alumina sheet and a pair of electrodes are provided on both surfaces of the zirconia filling part. Further, U.S. Pat. No. 5,198,832 discloses a gas sensor including a multilayer detector element, and the detector element includes a plate-like sensor function part having a solid electrolyte layer of which the main component is zirconia and a first part and a second part, each having a plate-like shape, which are layered on both surfaces of the sensor function part and each formed of a base layer of which the main component is alumina. In the gas sensor, the base layer of the first part and that of the second part have almost the same thickness, and on at least part of the detector element, provided is a symmetric structural part having a symmetric structure with respect to the solid electrolyte layer of the sensor function part in a layer-stacking direction. It is thereby possible to suppress a warp of the whole element.

Further, Japanese Patent Application Laid-Open No. 8-15213 discloses a technique used in an oxygen sensor with heater which is provided in an internal combustion engine exhaust system, and this technique is used for energizing the heater of the oxygen sensor on the condition of reaching a predetermined load amount which corresponds to a no-moisture generation temperature of an internal combustion engine exhaust pipe. By using this technique, it is possible to prevent an element breakage which is caused when water droplets in the exhaust pipe come into contact with a sensor element.

In a case where in the manufacture of a sensor element, or the like, a ceramic layered body in which two alumina layer parts are formed on both surfaces of a zirconia layer part is formed, a large warp occurs in the ceramic layered body. In such a case, for example, some trouble is disadvantageously caused in an assembly of a sensor using the sensor element, or the like.

### SUMMARY OF INVENTION

The present invention is intended for a sensor element, and it is an object of the present invention to suppress a warp of a ceramic layered body in a sensor element.

The sensor element according to the present invention includes a ceramic layered body having a zirconia layer part and two alumina layer parts provided on both surfaces of the zirconia layer part, respectively and a plurality of electrodes provided in the ceramic layered body. At least one alumina layer part out of the two alumina layer parts contains Ti element, the zirconia layer part has a layer containing Zr element and Ti element in the vicinity of an interface with the at least one alumina layer part, and the layer contains Ti element in an amount from 0.05 to 5.0 mass %.

According to the present invention, it is possible to suppress a warp of the ceramic layered body in the sensor element.

In one preferred embodiment of the present invention, the layer has a thickness of 5 to 100 μm.

In another preferred embodiment of the present invention, the at least one alumina layer part further contains another element included in any one of a transition metal group, a rare earth group, an alkali metal group, and an alkaline earth metal group.

In still another preferred embodiment of the present invention, both the two alumina layer parts each have Ti element.

In yet another preferred embodiment of the present invention, the zirconia layer part and the two alumina layer parts are formed by co-sintering.

In a further preferred embodiment of the present invention, the sensor element further includes a porous protection part covering part of the ceramic layered body.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is a view showing a gas sensor;

FIG. 2 is a cross section showing a structure of a sensor element;

FIG. 3 is a cross section showing the vicinity of an interface between an alumina layer part and a zirconia layer part;

FIG. 4 is a view showing a ceramic layered body; and

FIG. 5 is a view showing a warped ceramic layered body.

### DESCRIPTION OF EMBODIMENTS

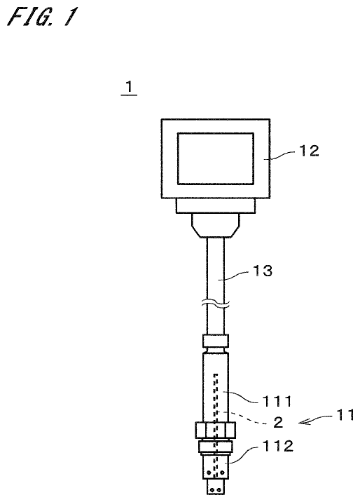

FIG. 1 is a view showing a gas sensor 1 in accordance with one preferred embodiment of the present invention. The gas sensor 1 is used for measuring the concentration of a predetermined gas component contained in a gas to be measured. As one example, the gas sensor 1 is used for measuring the concentration of nitrogen oxide (NOx) or the like contained in exhaust gas from an automobile. When the gas to be measured is exhaust gas, the gas sensor 1 is attached to, for example, an exhaust gas pipe of the automobile.

The gas sensor 1 includes a sensor body 11, an external connection part 12, and a tube 13. The tube 13 covers a plurality of lead wires for connecting the sensor body 11 to the external connection part 12. The external connection part 12 includes a plurality of terminal electrodes (not shown) connected to the plurality of lead wires, respectively. The terminal electrode is conducted with an electrode of a later-described sensor element 2 through the lead wire. The external connection part 12 is connected to, for example, a control unit of the automobile. The control unit supplies a current to the sensor element 2 and receives a signal from the sensor element 2.

The sensor body 11 includes the sensor element 2, a body tubular part 111, and a protective cover 112. The sensor element 2 has a long-length plate-like shape and measures the concentration of a predetermined gas component from the gas to be measured. The structure of the sensor element 2 will be described later. The body tubular part 111 is a tubular member accommodating the sensor element 2 thereinside. One end portion of the sensor element 2 (a lower end portion in FIG. 1, and hereinafter, referred to as a “tip portion”) is arranged outside the body tubular part 111, and the protective cover 112 surrounds the periphery of the tip portion of the sensor element 2. In the protective cover 112, formed is a through hole for passing the gas to be measured therethrough.

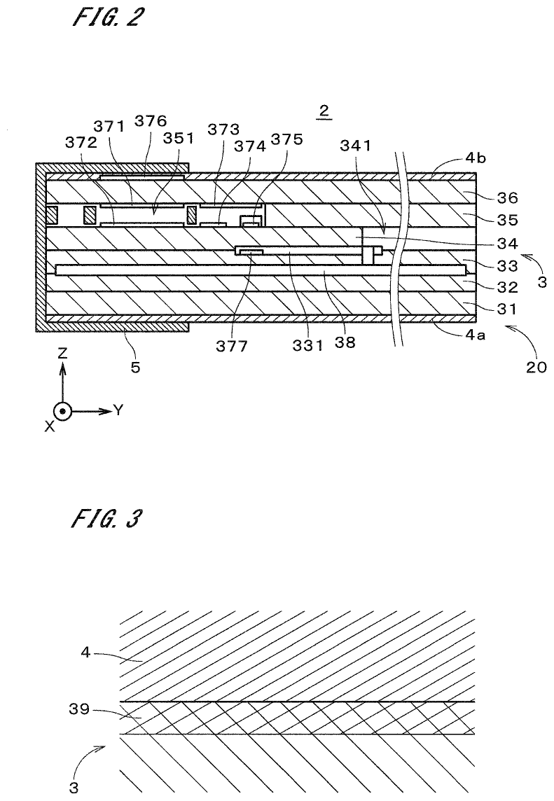

FIG. 2 is a cross section showing a structure of the sensor element 2. In FIG. 2, an X direction, a Y direction, and a Z direction which are orthogonal to one another are represented by arrows. The sensor element 2 has a long-length plate-like shape as described earlier, and the Y direction in FIG. 2 is a longitudinal direction of the sensor element 2 and the X direction is a width direction of the sensor element 2. Further, as described later, the sensor element 2 is formed by stacking a plurality of layers (or sheets), and the Z direction in FIG. 2 is a layer-stacking direction. FIG. 2 shows a cross section perpendicular to the width direction.

The sensor element 2 includes an element body 20 and a porous protection part 5 which covers part of the element body 20. The element body 20 includes a zirconia layer part 3 and two alumina layer parts 4a and 4b. In the element body 20, the two alumina layer parts 4a and 4b are provided on both surfaces (surfaces orienting in the layer-stacking direction) of the zirconia layer part 3, respectively. As described later, the zirconia layer part 3 and the alumina layer parts 4a and 4b are each mainly formed of ceramics, and the element body 20 is a ceramic layered body.

The zirconia layer part 3 includes a first substrate layer 31, a second substrate layer 32, a third substrate layer 33, a first solid electrolyte layer 34, a spacer layer 35, and a second solid electrolyte layer 36. The first substrate layer 31, the second substrate layer 32, the third substrate layer 33, the first solid electrolyte layer 34, the spacer layer 35, and the second solid electrolyte layer 36 are layered in this order from the (−Z) side toward the (+Z) direction.

The plurality of layers 31 to 36 included in the zirconia layer part 3 are each formed of ceramics of which the main component is zirconia (ZrO₂). Herein, the main component of each of the layers 31 to 36 refers to a component contained in the whole layer 31 to 36 in an amount of 50 mass % or more. The same applies to the following. Each of the layers 31 to 36 has a dense structure and has hermeticity. The zirconia layer part 3 (and each of the layers 31 to 36) of which the main component is zirconia has oxygen ion conductivity. In terms of more reliably displaying the oxygen ion conductivity in the zirconia layer part 3, the zirconia layer part 3 preferably contains zirconia in an amount of 65 mass % or more with respect to the whole of the zirconia layer part 3, and more preferably 80 mass % or more. As described later, the zirconia layer part 3 is formed by, for example, performing a predetermined processing, printing patterns, and the like on respective ceramic green sheets corresponding to the layers 31 to 36, layering these sheets, and sintering these sheets to be unified.

In the zirconia layer part 3, at a portion on the tip portion side ((−Y) side), a space 351 is formed by removing part of the spacer layer 35, and a plurality of electrodes 371 to 375 are provided in the space 351. Further, an electrode 376 is also formed on a surface of the second solid electrolyte layer 36 on the (+Z) side. Around the electrode 376, provided is a through hole for emitting oxygen pumped from the gas to be measured, to the outside. In the zirconia layer part 3, at a portion away from the tip portion toward the (+Y) side, a space 341 is provided between the third substrate layer 33 and the spacer layer 35. The space 341 is sectioned by a side surface of the first solid electrolyte layer 34. In the vicinity of the space 341, a porous ceramic layer 331 and an electrode 377 are provided between the third substrate layer 33 and the first solid electrolyte layer 34. Among these electrodes 371 to 377, at least some of the electrodes are each formed as a porous cermet electrode (e.g., a cermet electrode formed of platinum (Pt) and ZrO₂).

The zirconia layer part 3 further includes a heater part 38. The heater part 38 is provided between the second substrate layer 32 and the third substrate layer 33. The heater part 38 is formed by covering an electrical resistor with an insulative material such as alumina or the like. The electrical resistor is supplied with a current by a not-shown connector electrode. The oxygen ion conductivity in the solid electrolyte layers 34 and 36 is increased by heating the zirconia layer part 3 with the heater part 38, for example, to 600° C. or more.

In the zirconia layer part 3, an electrochemical pump cell and an electrochemical sensor cell are implemented by the electrodes 371 to 377 and the solid electrolyte layers 34 and 36. The gas to be measured is introduced from a not-shown gas introduction port into the above-described space 351, and the NOx concentration of the gas to be measured is measured by cooperation of the pump cell and the sensor cell. Thus, in the sensor element 2, the measurement using the oxygen ion conductivity in the zirconia layer part 3 is performed. Further, since the principle of the measurement of the NOx concentration in the sensor element 2 is well known, description thereof is omitted here.

The number of layers 31 to 36 described above in the zirconia layer part 3 may be changed as appropriate in accordance with the design of the sensor element 2. Typically, the zirconia layer part 3 includes a plurality of layers of which the main component is zirconia. In terms of easily manufacturing the element body 20, the lower limit value of the thickness of the zirconia layer part 3 in the layer-stacking direction is, for example, 400 μm, and preferably 500 In terms of reducing the size of the element body 20, the upper limit value of the thickness of the zirconia layer part 3 is, for example, 1800 and preferably 1600 μm.

The alumina layer part 4a is in contact with a surface of the first substrate layer 31 on the (−Z) side, and typically covers the entire surface. The alumina layer part 4b is in contact with a surface of the second solid electrolyte layer 36 on the (+Z) side, and typically covers the entire surface. The two alumina layer parts 4a and 4b are each formed of ceramics of which the main component is alumina (Al₂O₃). The alumina layer parts 4a and 4b protect the zirconia layer part 3. In terms of ensuring the strength in the alumina layer parts 4a and 4b to some degree, each of the alumina layer parts 4a and 4b preferably contains alumina in an amount of 65 mass % or more with respect to the whole of the alumina layer part 4a or 4b, and more preferably 80 mass % or more.

In terms of easily manufacturing the element body 20, the lower limit value of the thickness of each of the alumina layer parts 4a and 4b in the layer-stacking direction is, for example, 10 μm, preferably 20 μm, and more preferably 30 μm. In terms of reducing the size of the element body 20, the upper limit value of the thickness of each of the alumina layer parts 4a and 4b is, for example, 700 μm, preferably 600 μm, and more preferably 500 μm. Preferably, the respective thicknesses of the two alumina layer parts 4a and 4b are almost equal, and for example, the thickness of one of the alumina layer parts is not less than 80% and not more than 120% of that of the other alumina layer part. Depending on the design of the element body 20, the respective thicknesses of the two alumina layer parts 4a and 4b may be different from each other beyond the above range.

The lower limit value of the ratio (T1/T2) of the thickness T1 of the zirconia layer part 3 to the thickness T2 of each of the alumina layer parts 4a and 4b is, for example, 0.1, preferably 0.2, and more preferably 0.4. The upper limit value of the above ratio is, for example, 25, preferably 24, and more preferably 23. In terms of ensuring the strength in the alumina layer parts 4a and 4b to some degree, the upper limit value of the open porosity of the alumina layer parts 4a and 4b is, for example, 10%, and preferably 5%. The lower limit value of the open porosity of the alumina layer parts 4a and 4b is, for example, 0.1%, and preferably 0.3%. The open porosity can be measured by, for example, the Archimedes' method. Details of the material of the alumina layer parts 4a and 4b will be described later.

As described earlier, the sensor element 2 includes the porous protection part 5. The porous protection part 5 covers surfaces of a portion of the element body 20 on the tip portion side ((−Y) side). Specifically, the porous protection part 5 covers a tip portion side of a surface of the element body 20 on the (−Z) side, a tip portion side of a surface thereof on the (+Z) side, a tip portion side of a surface thereof on the (−X) side, a tip portion side of a surface thereof on the (+X) side, and an entire surface thereof on the (−Y) side. The porous protection part 5 is formed of porous ceramics such as alumina, zirconia, spinel, cordierite, titania, magnesia, or the like. In the present preferred embodiment, the porous protection part 5 is formed of alumina. In this case, since the alumina layer parts 4a and 4b and the porous protection part 5 each contain alumina, the adhesion between both the parts can be increased.

The porous protection part 5 protects a portion of the element body 20 on the tip portion side. If any moisture or the like contained in the gas to be measured is deposited onto the zirconia layer part 3, a deposited portion is locally cooled sharply, and the zirconia layer part 3 thereby receives thermal shock and there is a possibility that a crack may occur. On the other hand, in the sensor element 2 provided with the porous protection part 5, it is possible to prevent any moisture or the like contained in the gas to be measured from being deposited onto the zirconia layer part 3 and to suppress occurrence of the crack in the zirconia layer part 3. Further, with the porous protection part 5, it is possible to prevent an oil component or the like contained in the gas to be measured from being deposited onto the electrodes on the surface of the element body 20 and to suppress degradation of the electrodes. Furthermore, in the sensor element 2, the above-described gas introduction port in the zirconia layer part 3 is covered with the porous protection part 5, but since the porous protection part 5 is formed of porous body, the gas to be measured can pass through the porous protection part 5 and reach the gas introduction port.

In terms of appropriately protecting the element body 20, the lower limit value of the thickness of the porous protection part 5 is, for example, 100 μm, and preferably 200 μm. In terms of reducing the size of the sensor element 2, the upper limit value of the thickness of the porous protection part 5 is, for example, 1000 μm, and preferably 900 μm. In terms of appropriately introducing the gas to be measured to the gas introduction port of the zirconia layer part 3, the lower limit value of the open porosity of the porous protection part 5 is, for example, 5%, and preferably 10%. In terms of ensuring the strength in the porous protection part 5 to some degree, the upper limit value of the open porosity of the porous protection part 5 is, for example, 85%, and preferably 80%.

Next, details of the material of the alumina layer parts 4a and 4b will be described. In the following description, when the two alumina layer parts 4a and 4b are not distinguished from each other, the alumina layer parts 4a and 4b are generally referred to as the “alumina layer part 4”. The alumina layer part 4 contains alumina as the main component and further contains an additional element. Herein, the additional element refers to an element other than Al (aluminum) or O (oxygen) which is a constituent of alumina, and is an element included in any one of a transition metal group, a rare earth group, an alkali metal group, and an alkaline earth metal group (except Zr (zirconium), Y (yttrium), Mg (magnesium), and Ca (calcium)). The alumina layer part 4 may contain two or more kinds of elements included in any one of the transition metal group, the rare earth group, the alkali metal group, and the alkaline earth metal group.

A preferable additional element is any one element of Ti (titanium), Na (sodium), Sc (scandium), V (vanadium), Cr (chromium), Mn (manganese), Fe (iron), Ni (nickel), Cu (copper), Zn (zinc), Sr (strontium), Nb (niobium), Mo (molybdenum), Ba (barium), La (lanthanum), Ce (cerium), Pr (praseodymium), and Yb (ytterbium).

A more preferable additional element is Ti element. As one example, the alumina layer part 4 contains titania (TiO₂). The alumina layer part 4 may contain another element which is included in any one of the transition metal group, the rare earth group, the alkali metal group, and the alkaline earth metal group and different from Ti element, besides Ti element which is the additional element. As another element, Zr, Y, Mg, or Ca can be exemplarily used. As one example, any of these elements is present in the alumina layer part 4 as an oxide (zirconia, yttria, magnesia, or calcia) or as a composite oxide with Al or Ti. Further, a reaction layer 39 described later may contain another element described above. When the alumina layer part 4 contains Mg besides Ti element, the mechanical strength (herein, the bending strength) of the element body 20 can be increased.

In the element body 20 which is a ceramic layered body, since the alumina layer part 4 contains alumina as the main component and further contains the additional element, it is possible to suppress a warp of the element body 20, i.e., a warp of the sensor element 2. It is thereby possible to prevent any trouble in the assembly of the gas sensor 1 from occurring. Though the reason for suppressing the warp of the element body 20 is not necessarily clear, in the element body 20 in which the alumina layer part 4 contains the additional element, a layer 39 of reaction phase (hereinafter, referred to as a “reaction layer 39”) containing Zr element and the additional element is formed in the vicinity of an interface between each of the alumina layer parts 4 and the zirconia layer part 3 as shown in FIG. 3. Herein, the reaction layer 39 is assumed to be part of the zirconia layer part 3. The reaction layer 39 is a layer in contact with the alumina layer part 4. In the element body 20, there is a possibility that the presence of the reaction layer 39 may contribute to suppression of the warp. It is thought that the thermal expansion coefficient of the reaction layer 39 takes a value between the thermal expansion coefficient of the alumina layer part 4 and that of a portion of the zirconia layer part 3 except the reaction layer 39, and in this case, the reaction layer 39 alleviates the difference in the thermal expansion between the alumina layer part 4 and the zirconia layer part 3.

The thickness of the reaction layer 39 is sufficiently smaller than that of each of the layers 31 and 36 which are in contact with the alumina layer part 4, and preferably 5 to 100 μm. When the thickness of the reaction layer 39 becomes larger than 100 μm, there is a possibility that the oxygen ion conductivity of the zirconia layer part 3 may decrease. When the thickness of the reaction layer 39 becomes smaller than 5 μm, there is a possibility that the warp of the element body 20 may become larger or the zirconia layer part 3 and the alumina layer part 4 may be separated from each other. The thickness of the reaction layer 39 is more preferably 10 to 50 μm. For identifying the reaction layer 39, for example, the side surface of the element body 20 (surface along the layer-stacking direction) is mirror-polished and a surface analysis using the energy dispersive X-ray spectrometer (EDS) is performed on the polished surface. Then, a region in which Zr element and the additional element are mixed is identified as the reaction layer 39. Further, the thickness of the region is acquired as the thickness of the reaction layer 39. As a general rule, in the layers 31 and 36 of the zirconia layer part 3, which are in contact with the alumina layer part 4, a portion except the reaction layer 39 does not contain the additional element (Ti element in the preferable example), and in other words, the layers 31 and 36 each include a layer in which no additional element is present.

In the exemplary case where the additional element is Ti element, formed is the reaction layer 39 which uniformly contains Zr element and Ti element. As one example, the reaction layer 39 is formed, in which Ti element is solid-solved in a crystal structure of zirconia in the zirconia layer part 3. In the reaction layer 39, the crystal of titania may be mixed. The reaction layer 39 has only to be a layer containing Zr element and Ti element. The reaction layer 39 preferably contains Ti element in an amount from 0.05 to 5.0 mass %, and more preferably in an amount from 0.05 to 3.5 mass %. It is thereby possible to more reliably suppress a warp of the element body 20. For further suppressing a warp by forming the reaction layer 39 in which Ti element is appropriately dispersed, it is preferable that the percentage of Ti element in the reaction layer 39 should be not less than 0.1 mass %. Further, in order to increase the strength of the element body 20, it is preferable that the percentage of Ti element in the reaction layer 39 should be not more than 3.0 mass %. The percentage of Ti element in the reaction layer 39 can be acquired, for example, by the surface analysis using the above-described EDS. Through diffusion of Ti element contained in the alumina layer part 4 into the zirconia layer part 3 (the reaction layer 39), the mass percentage of Ti element in the alumina layer part 4 sometimes becomes lower locally in the vicinity of the reaction layer 39 than that in other portions. In other words, in the alumina layer part 4, the layer in which the mass percentage of Ti element is lower than that in other portions is sometimes provided in the vicinity of an interface with the reaction layer 39. In forming the reaction layer 39, Zr element may be diffused into the alumina layer part 4.

In the case where the additional element is Ti element, it is preferable that the alumina layer part 4 should contain Ti element in an amount of 0.1 mass % or more in terms of oxide (typically, as TiO₂). It is thereby possible to form the reaction layer 39 in which Ti element is appropriately dispersed and to more reliably suppress a warp of the element body 20. In order to form the reaction layer 39 in which Ti element is more uniformly dispersed, the alumina layer part 4 preferably contains Ti element in an amount of 0.5 mass % or more in terms of oxide, and more preferably in an amount of 1.0 mass % or more. Further, when the amount of Ti element contained in the alumina layer part 4 is excessively high, the amount of alumina for ensuring the mechanical strength disadvantageously becomes lower. Therefore, in order to ensure the mechanical strength in the element body 20 to some degree, the mass percentage of Ti element in the alumina layer part 4 is preferably 10 mass % or less in terms of oxide, more preferably 9 mass % or less, and further preferably 8 mass % or less.

Furthermore, depending on the design of the sensor element 2, there may be a case where the porous protection part 5 covering part of the element body 20 (the tip portion in the above-described exemplary case) is omitted and the part of the element body 20 is covered with the alumina layer part containing the additional element. In this case, in the element body 20 of FIG. 2, the alumina layer parts which cover the tip portion side of the surface on the (−X) side, the tip portion side of the surface on the (+X) side, and the entire surface on the (−Y) side, respectively, are formed, besides the alumina layer parts 4a and 4b. Since the alumina layer part has excellent water resistance, when any moisture or the like in the gas to be measured is deposited onto the element body 20, it is possible to suppress occurrence of a crack.

In the manufacture of the sensor element 2, first, the same number of unsintered ceramic green sheets as the number of layers 31 to 36 included in the zirconia layer part 3 are prepared. These ceramic green sheets are to become the above-described layers 31 to 36 and are zirconia green sheets each of which contains zirconia raw material as the main component. The zirconia green sheet contains an organic binder, an organic solvent, and the like, besides zirconia raw material (the same applies to an alumina green sheet described later). On each zirconia green sheet, printed are patterns of electrodes, an insulating layer, a resistance heating element, and the like in accordance with the design of the corresponding one of the layers 31 to 36.

Further, two unsintered ceramic green sheets are prepared. These ceramic green sheets are to become the alumina layer parts 4a and 4b and are alumina green sheets each of which contains alumina raw material as the main component and also contains the additional element. The additional element is contained in the alumina green sheet, for example, as an oxide such as titania or the like. Subsequently, with an adhesive paste interposed between the green sheets, one alumina green sheet, a plurality of zirconia green sheets corresponding to the above-described layers 31 to 36, and one alumina green sheet are layered in this order, to thereby form a layered body. The adhesive paste contains, for example, zirconia powder, the binder, and the organic solvent.

Typically, in the layered body, arranged are a plurality of element bodies in a state before being sintered. Each of the element bodies before being sintered is taken out by cutting the layered body and sintered at a predetermined sintering temperature (at the maximum temperature in sintering, and for example, 1300 to 1500° C.), to thereby obtain the element body 20. Thus, the zirconia layer part 3 and the two alumina layer parts 4a and 4b in the element body 20 are formed in a unified manner by co-sintering.

Further, an alumina sheet before being sintered may be formed by applying a paste containing alumina as the main component and the additional element onto surfaces of the zirconia green sheets which serve as both surfaces of the zirconia layer part 3. Furthermore, the element body 20 does not necessarily need to be formed by co-sintering, but may be formed in such a method, for example, where the zirconia layer part 3 and the alumina layer parts 4a and 4b are individually prepared by sintering and then the zirconia layer part 3 and the alumina layer parts 4a and 4b are layered with the adhesive paste interposed therebetween and sintered again.

After the element body 20 which is a sintered body is obtained, the porous protection part 5 is formed on part of the surfaces of the element body 20. The porous protection part 5 is formed, for example, by plasma spraying using a plasma gun. In the plasma spraying, for example, a thermal spray material containing alumina powder is sprayed together with a carrier gas onto surfaces of the element body 20 at a portion on the tip portion side ((−Y) side). Specifically, the thermal spray material is sprayed onto the tip portion side of the surface of the element body 20 on the (−Z) side, the tip portion side of the surface thereof on the (+Z) side, the tip portion side of the surface thereof on the (−X) side, the tip portion side of the surface thereof on the (+X) side, and the entire surface thereof on the (−Y) side, to thereby form the porous protection part 5. The sensor element 2 is thereby completed.

In the case where the element body 20 is formed by co-sintering, it is preferable that a sintering shrinkage curve of the alumina green sheets which are to become the alumina layer parts 4a and 4b and that of the zirconia green sheet which is to become the zirconia layer part 3 should be approximate to each other. Herein, the sintering shrinkage curve indicates a change in the shrinkage ratio (the ratio of the shrunk length to the initial length) of the green sheet with the temperature rise in sintering. Assuming a temperature at the time when the shrinkage ratio of the green sheet in the course of sintering is 2% or more as a shrinkage starting temperature, for example, when the difference (absolute value) between the shrinkage starting temperature of the alumina green sheet and that of the zirconia green sheet is approximate to some degree and the difference (absolute value) between the shrinkage ratio of the alumina green sheet and that of the zirconia green sheet at an actual sintering temperature is approximate to some degree, the two sintering shrinkage curves are approximate to each other. The sintering shrinkage curve (the shrinkage starting temperature and the shrinkage ratio at the sintering temperature) can be measured by using a thermomechanical analyzer (TMA).

In the case where the sintering shrinkage curve of the alumina green sheet and that of the zirconia green sheet are approximate to each other, at the temperature rise in co-sintering, the alumina green sheet and the zirconia green sheet start shrinkage almost the same time, and even when the temperature reaches the sintering temperature (maximum temperature), both sheets have almost the same amount of shrinkage. Therefore, it becomes possible to further suppress a warp of the element body 20. For example, the sintering shrinkage curve of the alumina green sheet containing no Ti element is not approximate to that of the zirconia green sheet, but the sintering shrinkage curve of the alumina green sheet containing Ti element (e.g., titania) as the additional element is approximate to that of the zirconia green sheet. In order to more reliably suppress a warp of the element body 20, the difference between the shrinkage starting temperature of the alumina green sheet and that of zirconia green sheet is preferably 70° C. or less, more preferably 50° C. or less, and further preferably 30° C. or less. Further, though the difference between the shrinkage ratio of the alumina green sheet and that of the zirconia green sheet at the sintering temperature does not become large, in order to more reliably suppress a warp, the difference is preferably 4% points or less, more preferably 3% points or less, and further preferably 2% points or less.

In the case where the sintering shrinkage curve of the alumina green sheet is adjusted by the aid (additive) as described above, the element contained in the aid is sometimes diffused into the zirconia layer part 3 in the co-sintering process. In this case, depending on the kind of or the amount of aid, due to the diffusion of the element contained in the aid into the zirconia layer part 3, there is a possibility that some effect may be produced on the properties of the element body 20 (for example, the oxygen ion conductivity of the zirconia layer part 3 is reduced). In contrast to this, in the case where the alumina green sheet in which the aid containing Ti element is added in an appropriate amount so that the reaction layer 39 can contain Ti element in an amount from 0.05 to 5.0 mass % is used in the element body 20 which is a sintered body, it is possible to suppress a warp of the element body 20 in the co-sintering process while suppressing any effect from being produced on the properties of the element body 20.

### Examples

(Formation of Ceramic Layered Body)

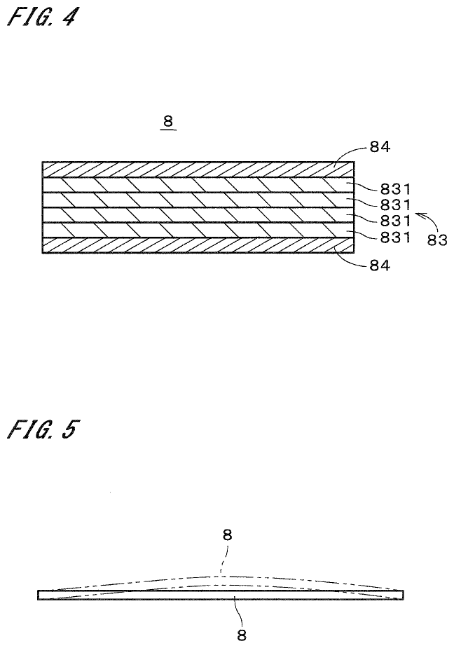

Next, Examples of the ceramic layered body will be described. Herein, a ceramic layered body 8 is formed in which a zirconia layer part 83 includes four layers 831 and two alumina layer parts 84 are formed on both surfaces of the zirconia layer part 83 as shown in FIG. 4.

In forming the ceramic layered body 8, first, powder of alumina, powder of titania serving as an aid, powder of another aid, a plasticizer, and an organic solvent are weighed, and these materials are mixed for 10 hours by using a pot mill. A mixture which is to become a raw material of the alumina green sheet is thereby obtained. The mixing ratio of alumina (Al₂O₃), titania (TiO₂), and other aids (SiO₂, ZrO₂, MgO, Y₂O₃) in the mixture is shown in the columns of “Composition” of Table 1.

TABLE 1

| | Composition [mass %] | | | | | |
|---|---|---|---|---|---|---|
| | Al₂O₃ | TiO₂ | SiO₂ | ZrO₂ | MgO | Y₂O₃ |
| Example 1 | 98 | 2 | — | — | — | — |
| Example 2 | 99.9 | 0.1 | — | — | — | — |
| Example 3 | 90 | 10 | — | — | — | — |
| Example 4 | 96 | 2 | — | — | 2 | — |
| Example 5 | 92 | 2 | — | — | 6 | — |
| Example 6 | 95 | 1 | — | — | 4 | — |
| Example 7 | 97 | 2 | — | 1 | — | — |
| Example 8 | 89 | 11 | — | — | — | — |
| Comparative | 100 | 0 | — | — | — | — |
| Example 1 | | | | | | |
| Comparative | 98 | — | 2 | — | — | — |
| Example 2 | | | | | | |
| Comparative | 98 | — | — | 2 | — | — |
| Example 3 | | | | | | |
| Comparative | 98 | — | — | — | 2 | — |
| Example 4 | | | | | | |
| Comparative | 98 | — | — | — | — | 2 |
| Example 5 | | | | | | |

Further, a binder solution containing a polyvinyl butyral (PVB) resin and the organic solvent is added to the above-described mixture and further mixed for 10 hours. After that, viscosity adjustment is performed by a predetermined method, and the alumina green sheet is obtained by tape molding. The thickness of the alumina green sheet is 250 Further, the zirconia green sheet containing zirconia raw material is obtained by the same operation as that for the alumina green sheet. The thickness of the zirconia green sheet is 250 μm.

Subsequently, the adhesive paste containing zirconia powder, the binder, and the organic solvent is applied onto the green sheet by screen printing. Then, with the adhesive paste interposed between the green sheets, one alumina green sheet, four zirconia green sheets, and one alumina green sheet are layered in this order, to thereby form a layered body. The thickness of the layered body is 1.5 mm. Further, printing of patterns of the electrodes and the like is omitted. After that, the layered body is cut to the size of (85 mm×5 mm) and sintered at 1400° C. The ceramic layered body 8 in each of Examples 1 to 8 is obtained. Further, the ceramic layered body 8 in each of Comparative Examples 1 to 5 is formed by the same operation as that for Examples. As shown in Table 1, in the ceramic layered body 8 in each of Comparative Examples 1 to 5, the alumina green sheet does not contain titania which is a raw material of the additional element.

Next, various measurements are performed on the ceramic layered bodies 8 of Examples 1 to 8 and Comparative Examples 1 to 5. Table 2 shows the measurement results.

TABLE 2

| | Measurement Result | | | | | | | |
|---|---|---|---|---|---|---|---|---|
| | | | | | | Percentage | Bending | |
| | | | | | Thickness of | of Ti Element | Strength | |
| | | Shrinkage | | | Reaction | in Reaction | (Maximum | Water |
| | Open | Starting | Warp | Reaction | Layer | Layer | Load) | Resistance |
| | Porosity | Temperature | [μm] | Layer | [μm] | [mass %] | [N] | [μL] |
| Example 1 | ◯ | ◯ | 200 | Present | 40 | 0.4 | 220 | 60 |
| Example 2 | ◯ | ◯ | 250 | Present | 5 | 0.05 | 210 | 60 |
| Example 3 | ◯ | ◯ | 230 | Present | 100 | 3.0 | 200 | 55 |
| Example 4 | ◯ | ⊚ | 140 | Present | 30 | 0.2 | 250 | 70 |
| Example 5 | ◯ | ⊚ | 120 | Present | 30 | 0.2 | 260 | 75 |
| Example 6 | ◯ | ⊚ | 170 | Present | 20 | 0.1 | 230 | 65 |
| Example 7 | ◯ | ◯ | 250 | Present | 30 | 0.2 | 200 | 50 |
| Example 8 | Δ | ◯ | 240 | Present | 100 | 3.5 | 150 | 5 |
| Comparative | Δ | Δ | 900 | Absent | 0 | 0 | 220 | 60 |
| Example 1 | | | | | | | | |
| Comparative | X | X | — | — | — | — | — | — |
| Example 2 | | | | | | | | |
| Comparative | Δ | X | — | — | — | — | — | — |
| Example 3 | | | | | | | | |
| Comparative | ◯ | Δ | 900 | Absent | 0 | 0 | 220 | 50 |
| Example 4 | | | | | | | | |
| Comparative | X | X | — | — | — | — | — | — |
| Example 5 | | | | | | | | |

(Measurement of Open Porosity)

The measurement of the open porosity is performed by the Archimedes' method, on the single alumina layer part 84 which is obtained by sintering the alumina green sheet. In the column of “Open Porosity” of Table 2, “◯ (circle)” is given to the ceramic layered body 8 in which the open porosity of the alumina layer part 84 is not lower than 0% and lower than 4%, “Δ (triangle)” is given to the ceramic layered body 8 in which the open porosity of the alumina layer part 84 is not lower than 4% and lower than 10%, and “X (cross)” is given to the ceramic layered body 8 in which the open porosity of the alumina layer part 84 is not lower than 10%. In the ceramic layered bodies 8 of Comparative Examples 2 and 5 each containing SiO₂ and Y₂O₃ as the aids, the open porosity of the alumina layer part 84 is not lower than 10% (the denseness becomes lower), and on the other hand, in the ceramic layered bodies 8 of Examples 1 to 8 and Comparative Examples 1, 3, and 4, the open porosity is lower than 10% and the alumina layer part 84 which is dense can be obtained.

(Measurement of Shrinkage Starting Temperature)

For the measurement of the shrinkage starting temperature, the shrinkage starting temperature in singly sintering the alumina green sheet in each of Examples 1 to 8 and Comparative Examples 1 to 5 is measured by using the thermomechanical analyzer (TMA). It is assumed that the shrinkage starting temperature is a temperature at the time when the shrinkage ratio of the green sheet becomes 2% or more. Further, the shrinkage starting temperature in singly sintering the zirconia green sheet is also measured, and the difference between the shrinkage starting temperature of the alumina green sheet and that of the zirconia green sheet is obtained. In the column of “Shrinkage Starting Temperature” of Table 2, “⊚ (double circle)” is given to the ceramic layered body 8 in which the absolute value of the difference between the shrinkage starting temperature of the alumina green sheet and that of the zirconia green sheet (hereinafter, referred to simply as the “difference in the shrinkage starting temperature”) is not higher than 30° C., “◯ (circle)” is given to the ceramic layered body 8 in which the difference in the shrinkage starting temperature is higher than 30° C. and not higher than 50° C., “Δ (triangle)” is given to the ceramic layered body 8 in which the difference in the shrinkage starting temperature is higher than 50° C. and not higher than 70° C., and “X (cross)” is given to the ceramic layered body 8 in which the difference in the shrinkage starting temperature is higher than 70° C. In Examples 1 to 8, the difference in the shrinkage starting temperature is not higher than 50° C., and on the other hand, in Comparative Examples 1 to 5, the difference in the shrinkage starting temperature is higher than 50° C. In Comparative Examples 2, 3, and 5, the difference in the shrinkage starting temperature is higher than 70° C., and in the ceramic layered body 8, the alumina layer part 84 and the zirconia layer part 83 are separated from each other. Therefore, for Comparative Examples 2, 3, and 5, the other measurements in Table 2 are not performed.

(Measurement of Warp)

In FIG. 5, a warped ceramic layered body 8 is represented by a two-dot chain line. For the measurement of the warp, in a state where one alumina layer part 84 is arranged on the lower side, the ceramic layered body 8 is placed on a horizontal placement surface, and an entire surface of the other alumina layer part 84, which faces upward, is scanned by using the 3D Measurement System (manufactured by Keyence Corporation, VR-3000). With the placement surface set as a reference plane in an average step mode, a region in a range of 80% or more of the above-described surface of the alumina layer part 84 in the longitudinal direction and in a range of 30% or more thereof in the width direction (short-side direction) is set as a measurement surface. Then, a value obtained by subtracting the minimum height from the maximum height of the measurement surface is calculated as the warp.

As shown in Table 2, in the ceramic layered body 8 of each of Examples 1 to 8, the warp is 300 μm or less, and on the other hand, in the ceramic layered body 8 of each of Comparative Examples 1 and 4, the warp largely exceeds 300 μm. When the warp of the ceramic layered body 8 exceeds 300 μm, in the case where the ceramic layered body 8 is the above-described element body 20, there occurs some trouble in the assembly of the gas sensor L Further, in the ceramic layered body 8 of each of Examples 4 to 6, the warp is less than 200 μm. In the ceramic layered body 8 of each of Examples 4 to 6, by adding MgO to the raw material of the alumina green sheet, it is thought that the difference in the shrinkage starting temperature becomes 30° C. or lower and the warp is significantly suppressed.

(Check of Reaction Layer and Various Measurements of Reaction Layer)

For the check of the reaction layer, after mirror-polishing the side surface (surface along the layer-stacking direction) of the ceramic layered body 8, the vicinity of an interface between the zirconia layer part 83 and the alumina layer part 84 in the polished surface is observed by using the scanning electron microscope (SEM) with a magnification of 1000 times. Further, the surface analysis of Zr and Ti is performed by using the energy dispersive X-ray spectrometer (EDS), and a region of the zirconia layer part 83 in which Ti element is present (region in which Zr element and Ti element are mixed) is identified as the reaction layer. For the analysis of Zr and Ti, the electron probe micro analyzer (EPMA) can be also used. As shown in Table 2, in the ceramic layered body 8 of each of Examples 1 to 8, the presence of the reaction layer can be confirmed, and on the other hand, in the ceramic layered body 8 of each of Comparative Examples 1 and 4, the presence of the reaction layer cannot be confirmed. Therefore, it is thought that the presence of the reaction layer contributes to suppression of the warp.

The thickness of the region identified as the reaction layer in the above-described check of the reaction layer, i.e., the region in which Zr element and Ti element are mixed is measured as the thickness of the reaction layer. In the ceramic layered body 8 of each of Examples 1 to 8, the thickness of the reaction layer is within a range from 5 to 100 μm. Further, from the surface analysis using the above-described EDS, the percentage of Ti element in the reaction layer is acquired. From Examples 1 to 8, when the percentage of Ti element in the reaction layer is 0.05 to 3.5 mass %, it is possible to more reliably suppress the warp. Also in the ceramic layered body 8 of Example 8 in which the percentage of Ti element in the reaction layer is 3.5 mass %, the warp is 240 μm and sufficiently small. Therefore, when the percentage of Ti element is 5.0 mass % or less, it is thought that the warp can be suppressed to 300 μm or less.

As is clear from Tables 1 and 2, the thickness of the reaction layer and the percentage of Ti element in the reaction layer depends on the mass percentage of TiO₂ in the raw material of the alumina green sheet. When the mass percentage of TiO₂ in the raw material of the alumina green sheet is excessively small, the thickness of the reaction layer and the percentage of Ti element each become sufficiently small, and in this case, it is thought that the warp becomes larger or the alumina layer part 84 and the zirconia layer part 83 are separated from each other. In other words, in the case where the thickness of the reaction layer is 5 μm or more or the percentage of Ti element in the reaction layer is 0.05 mass % or more, it is possible to more reliably suppress the separation and the warp from occurring.

(Measurement of Bending Strength)

For the measurement of the bending strength, the layered body before being sintered is cut so that the size of the layered body after being sintered can be (40 mm×4 mm) and the layered body is sintered in the same manner as that in the formation of the ceramic layered body 8, to thereby obtain a specimen. Then, the four-point bending strength of each specimen in the layer-stacking direction is measured by using the strength measurement instrument (manufactured by Instron Ltd.).

As shown in Table 2, in the specimen of each of Examples 1 to 7 and Comparative Examples 1 and 4, the breaking load in the bending test is 200 N or more, and on the other hand, in Example 8, the breaking load in the bending test is less than 200 N. Therefore, in order to ensure the mechanical strength in the ceramic layered body 8 to some degree, it is preferable that the mass percentage of Ti element in the alumina layer part 84 should be 10 mass % or less in terms of oxide or the percentage of Ti element in the reaction layer should be 3.0 mass % or less. It is thereby possible to prevent Al₂O₃ and ZrO₂ which take on the strength in the ceramic layered body 8 from being relatively reduced. Further, in the ceramic layered body 8 of each of Examples 4 to 6, by adding MgO to the raw material of the alumina green sheet, the mechanical strength is further increased (the same applies to the water resistance described later).

(Measurement of Water Resistance) For the measurement of the water resistance, the ceramic layered body 8 is placed on a heater and heated to 800° C. When the surface temperature of the ceramic layered body 8 becomes 800° C., by dropping a predetermined amount of water droplet, whether or not a crack occurs in the ceramic layered body 8 is visually checked. Until a crack occurs, the above operation is repeated while the amount of water droplet is increased.

As shown in Table 2, in the ceramic layered body 8 of each of Examples 1 to 7 and Comparative Examples 1 and 4, the amount of water droplet needed to cause a crack is 50 μL or more, and on the other hand, in Example 8, a crack occurs with 5 μL of water droplet. Though the reason for decreasing the water resistance in the ceramic layered body 8 of Example 8 is not clear, in order to ensure the water resistance in the ceramic layered body 8 to some degree, it is thought that it is preferable that the mass percentage of Ti element in the alumina layer part 84 should be 10 mass % or less in terms of oxide or the percentage of Ti element in the reaction layer should be 3.0 mass % or less, like in the case of the mechanical strength.

In the sensor element and the ceramic layered body described above, various modifications can be made.

Though both the two alumina layer parts 4a and 4b each contain the additional element (e.g., Ti element) in the above-described element body 20 (and the ceramic layered body), even in a case where one of the alumina layer parts contains the additional element and the other alumina layer part does not contain any additional element, a warp of the element body 20 can be suppressed to some degree. As described above, when at least one of the two alumina layer parts contains the additional element (e.g., Ti element) in the element body 20, it is possible to suppress a warp of the element body 20. Further, it is preferable that the zirconia layer part 3 should have the reaction layer 39 containing Zr element and the additional element in the vicinity of an interface with the at least one alumina layer part.

The sensor element 2 may be used for any sensor other than the gas sensor 1. The ceramic layered body in which a warp is suppressed by using the additional element may be used for any use other than the sensor element 2. For example, the above-described ceramic layered body can be used as a sintering setter requiring high thermal shock residence. Depending on the use of the ceramic layered body, the zirconia layer part may include only one layer of which the main component is zirconia. Further, each alumina layer part may include a plurality of layers in each of which the main component is alumina. Thus, in the ceramic layered body, the zirconia layer part has only to include one or a plurality of layers of which the main component is zirconia and the alumina layer part has only to include one or a plurality of layers of which the main component is alumina.

The configurations in the above-discussed preferred embodiment and variations may be combined as appropriate only if those do not conflict with one another.

While the invention has been shown and described in detail, the foregoing description is in all aspects illustrative and not restrictive. It is therefore understood that numerous modifications and variations can be devised without departing from the scope of the invention.

### REFERENCE SIGNS LIST

2 Sensor element
3, 83 Zirconia layer part
4, 4a, 4b, 84 Alumina layer part
5 Porous protection part
8 Ceramic layered body
20 Element body
39 Reaction layer
371 to 377 Electrode

1. A sensor element, comprising:
a ceramic layered body having a zirconia layer part and two alumina layer parts provided on both surfaces of said zirconia layer part, respectively; and
a plurality of electrodes provided in said ceramic layered body,
wherein at least one alumina layer part out of said two alumina layer parts contains Ti element,
said zirconia layer part has a layer containing Zr element and Ti element in the vicinity of an interface with said at least one alumina layer part, and
said layer contains Ti element in an amount from 0.05 to 5.0 mass %.

2. The sensor element according to claim 1, wherein
said layer has a thickness of 5 to 100 μm.

3. The sensor element according to claim 1, wherein
said at least one alumina layer part further contains another element included in any one of a transition metal group, a rare earth group, an alkali metal group, and an alkaline earth metal group.

4. The sensor element according to claim 1, wherein
both said two alumina layer parts each have Ti element.

5. The sensor element according to claim 1, wherein
said zirconia layer part and said two alumina layer parts are formed by co-sintering.

6. The sensor element according to claim 1, further comprising:
a porous protection part covering part of said ceramic layered body.
